(19) United States
(12) Patent Application Publication
(10) Pub. No.: US 2013/0282091 A1
(43) Pub. Date: Oct. 24, 2013

(54) SYSTEMS AND METHODS FOR MAKING AND USING IMPROVED ELECTRODES FOR IMPLANTABLE PADDLE LEADS

(71) Applicant: BOSTON SCIENTIFIC NEUROMODULATION CORPORATION, (US)

(72) Inventor: Jacob B. Leven, Huntington Beach, CA (US)

(73) Assignee: BOSTON SCIENTIFIC NEUROMODULATION CORPORATION, Valencia, CA (US)

(21) Appl. No.: 13/867,704

(22) Filed: Apr. 22, 2013

Related U.S. Application Data
(60) Provisional application No. 61/637,182, filed on Apr. 23, 2012.

(51) Int. Cl. A61N 1/05

(52) U.S. Cl. CPC A61N 1/0553
USPC 607/116

(57) ABSTRACT

A paddle lead assembly for providing electrical stimulation of patient tissue includes a paddle body having a longitudinal axis and a transverse axis transverse to the longitudinal axis. A plurality of electrodes are disposed along the paddle body. Each of the plurality of electrodes has a five-sided shape. At least one lead body is coupled to the paddle body. A plurality of terminals are disposed on the at least one lead body. The paddle lead assembly further includes a plurality of conductive wires. Each conductive wire couples one of the plurality of terminals to at least one of the plurality of electrodes.

### CROSS-REFERENCE TO RELATED APPLICATIONS

This application claims the benefit under 35 U.S.C. §119(e) of U.S. Provisional Patent Application Ser. No. 61/637,182 filed on Apr. 23, 2012, which is incorporated herein by reference.

### FIELD

The present invention is directed to the area of implantable electrical stimulation systems and methods of making and using the systems. The present invention is also directed to implantable leads with paddle bodies that include electrodes configured to improve flexibility of the paddle bodies, as well as methods of making and using the leads, paddle bodies, electrodes, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a paddle lead assembly for providing electrical stimulation of patient tissue includes a paddle body having a longitudinal axis and a transverse axis transverse to the longitudinal axis. A plurality of electrodes are disposed along the paddle body. Each of the plurality of electrodes has a five-sided shape. At least one lead body is coupled to the paddle body. A plurality of terminals is disposed on the at least one lead body. The paddle lead assembly further includes a plurality of conductive wires. Each conductive wire couples one of the plurality of terminals to at least one of the plurality of electrodes.

In another embodiment, a paddle lead assembly for providing electrical stimulation of patient tissue includes a paddle body having a longitudinal axis and a transverse axis transverse to the longitudinal axis. A plurality of multi-sided electrodes are disposed along the paddle body with each electrode of the plurality of electrodes having a first edge portion and a second edge portion. For each electrode of the plurality of electrodes the first edge portion abuts the first edge portion of another electrode of the plurality of electrodes along a first interface. The first interface extends in a direction that is parallel to neither the longitudinal axis nor the transverse axis of the paddle body. At least one lead body is coupled to the paddle body. A plurality of terminals are disposed on the at least one lead body. The paddle lead assembly further includes a plurality of conductive wires. Each conductive wire couples one of the plurality of terminals to at least one of the plurality of electrodes.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle body coupled to a control module via lead bodies, according to the invention;

FIG. 2A is a schematic side view of one embodiment of a plurality of connector assemblies disposed in the control module of FIG. 1, the connector assemblies configured and arranged to receive the proximal portions of the lead bodies of FIG. 1, according to the invention;

FIG. 2B is a schematic side view of one embodiment of a proximal portion of a lead body and a lead extension coupled to a control module, the lead extension configured and arranged to couple the proximal portion of the lead body to the control module, according to the invention;

FIG. 2C is a schematic side view of one embodiment of a connector assembly disposed in the control module of FIG. 2B, the connector assembly configured and arranged to receive the lead extension of FIG. 2B, according to the invention;

FIG. 3 is a schematic longitudinal cross-sectional view of one embodiment of one of the connector assemblies of FIG. 1, according to the invention;

FIG. 4 is a schematic perspective view a control module with a header that defines four ports, according to the invention;

FIG. 5 is a schematic top view of one embodiment of a paddle lead assembly that includes a paddle body with four columns of five-sided electrodes, according to the invention;

FIG. 6 is a schematic top view of one embodiment of a five-sided electrode suitable for use with the paddle lead assembly of FIG. 5, according to the invention;

FIG. 7 is a schematic top close-up view of a portion of the paddle lead assembly of FIG. 5, the portion of the paddle lead assembly including five-sided electrodes disposed on a portion of a paddle body, each of the electrodes having a double-sided face that abuts a portion of a double-sided face of at least one other electrode, according to the invention;

FIG. 8A is a schematic view of a first exemplary embodiment of a five-sided electrode suitable for use with the paddle lead assembly of FIG. 5, the electrode having a single-sided face with a length that is greater than a length of either a first end or an opposing second end of the electrode, according to the invention;

FIG. 8B is a schematic view of a second exemplary embodiment of a five-sided electrode suitable for use with the paddle lead assembly of FIG. 5, the electrode having a single-sided face with a length that is greater than a length of either a first end or an opposing second end of the electrode, according to the invention;

FIG. 8C is a schematic view of a third exemplary embodiment of a five-sided electrode suitable for use with the paddle lead assembly of FIG. 5, the electrode having a single-sided face with a length that greater than a length of either a first end or an opposing second end of the electrode, according to the invention;

FIG. 8D is a schematic view of a fourth exemplary embodiment of a five-sided electrode suitable for use with the paddle lead assembly of FIG. 5, the electrode having a single-sided face with a length that is greater than a length of either a first end or an opposing second end of the electrode, according to the invention;

FIG. 9 is a schematic top view of one embodiment of a paddle lead assembly that includes the electrodes of FIG. 8D disposed on a paddle body, according to the invention; and

FIG. 10 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable leads with paddle bodies that include electrodes configured to improve flexibility of the paddle bodies, as well as methods of making and using the leads, paddle bodies, electrodes, and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; 7,974,706; 8,175,710; 8,224,450; and 8,364,278; and U.S. Patent Application Publication No. 2007/0150036, all of which are incorporated by reference.

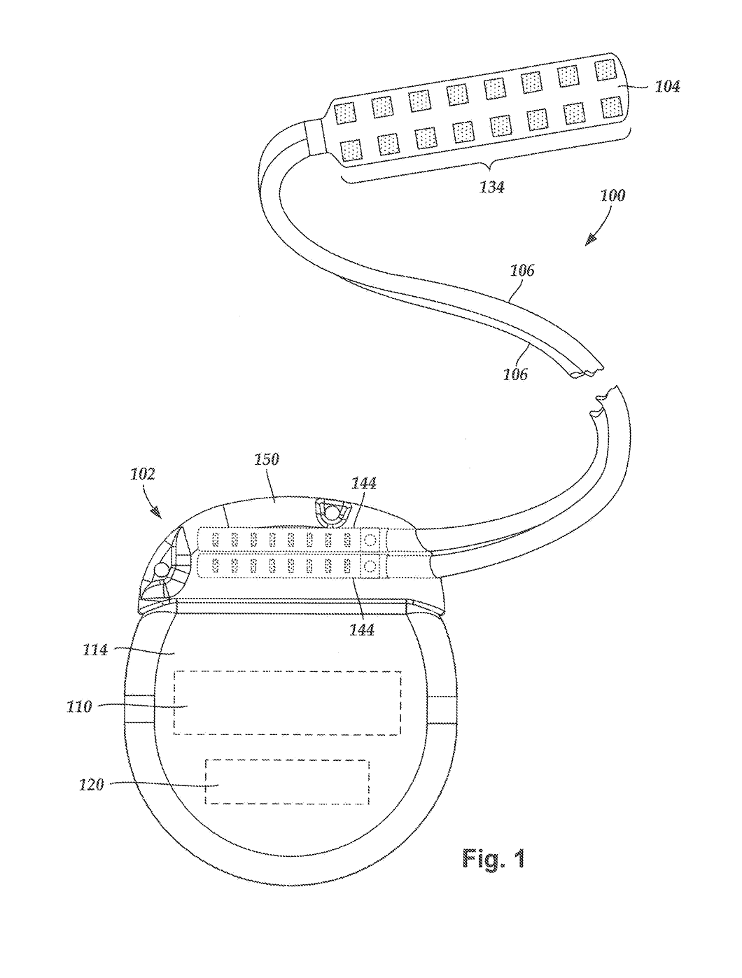

FIG. 1 illustrates schematically one embodiment of an electrical stimulation system 100. The electrical stimulation system includes a control module (e.g., a stimulator or pulse generator) 102, a paddle body 104, and one or more lead bodies 106 coupling the control module 102 to the paddle body 104. The paddle body 104 and the one or more lead bodies 106 form a lead. The paddle body 104 typically includes an array of electrodes 134. The control module 102 typically includes an electronic subassembly 110 and an optional power source 120 disposed in a sealed housing 114. In FIG. 1, two lead bodies 106 are shown coupled to the control module 102.

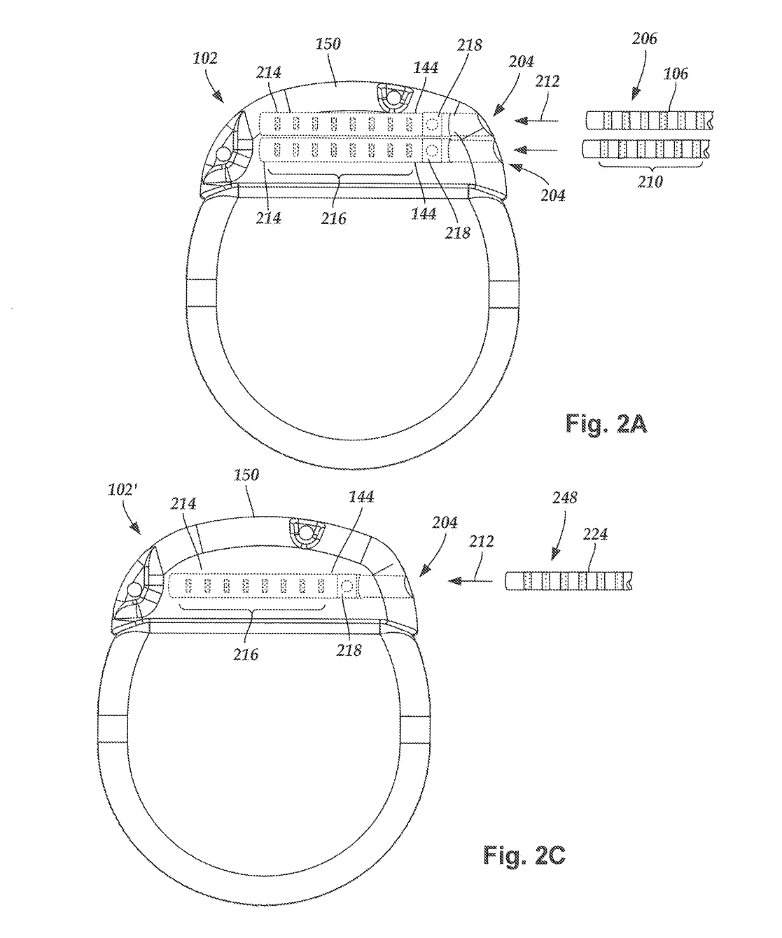

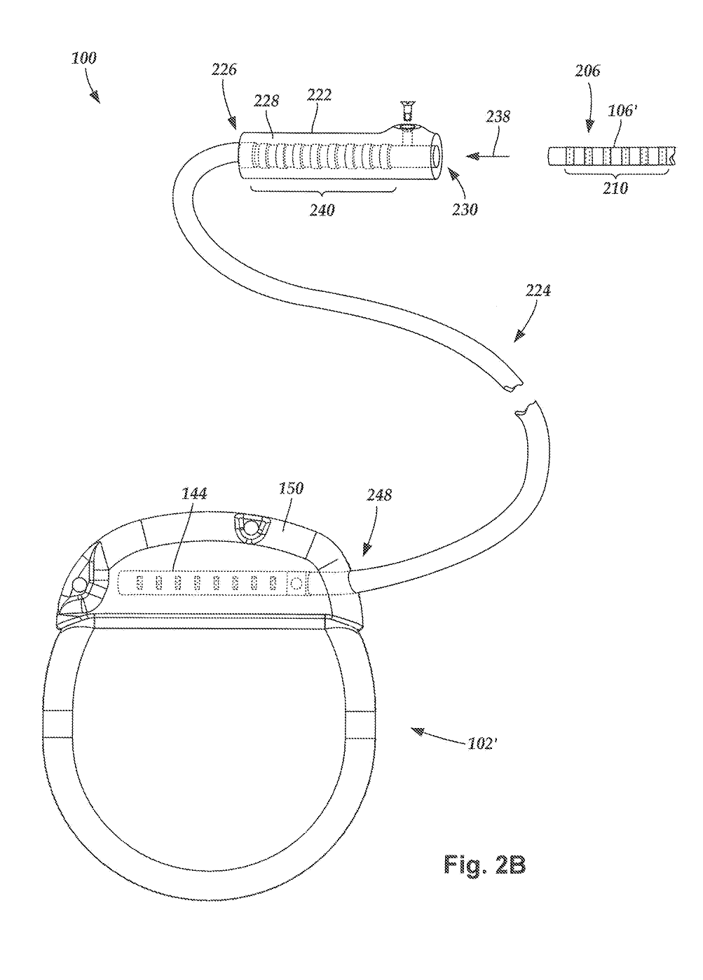

The control module 102 typically includes one or more connector assemblies 144 into which the proximal end of the one or more lead bodies 106 can be plugged to make an electrical connection via connector contacts (e.g., 216 in FIG. 2A). The connector contacts are coupled to the electronic subassembly 110 and the terminals are coupled to the electrodes 134. In FIG. 1, two connector assemblies 144 are shown.

The one or more connector assemblies 144 may be disposed in a header 150. The header 150 provides a protective covering over the one or more connector assemblies 144. The header 150 may be formed using any suitable process including, for example, casting, molding (including injection molding), and the like. In addition, one or more lead extensions 224 (see FIG. 2B) can be disposed between the one or more lead bodies 106 and the control module 102 to extend the distance between the one or more lead bodies 106 and the control module 102.

The electrical stimulation system or components of the electrical stimulation system, including one or more of the lead bodies 106, the paddle body 104, and the control module 102, are typically implanted into the body of a patient. The electrical stimulation system can be used for a variety of applications including, but not limited to, spinal cord stimulation, brain stimulation, neural stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, titanium nitride, or rhenium.

The number of electrodes 134 in the array of electrodes 134 may vary. For example, there can be two, three, four, five, six, seven, eight, nine, ten, eleven, twelve, thirteen, fourteen, fifteen, sixteen, or more electrodes 134. As will be recognized, other numbers of electrodes 134 may also be used. As will be recognized, other numbers of electrodes 134 may also be used. In FIG. 1, sixteen electrodes 134 are shown. The electrodes 134 can be formed in any suitable shape including, for example, round, oval, triangular, rectangular, pentagonal, hexagonal, heptagonal, octagonal, or the like.

The electrodes of the paddle body 104 or one or more lead bodies 106 are typically disposed in, or separated by, a non-conductive, biocompatible material including, for example, silicone, polyurethane, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 210 in FIG. 2A) are typically disposed at the proximal end of the one or more lead bodies 106 for connection to corresponding conductive contacts (e.g., 216 in FIG. 2A) in connector assemblies disposed on, for example, the control module 102 (or to other devices, such as conductive contacts on a lead extension, an operating room cable, a splitter, an adaptor, or the like). Conductive wires (not shown) extend from the terminals to the electrodes 134. Typically, one or more electrodes 134 are electrically coupled to a terminal (e.g., 210 in FIG. 2A). In some embodiments, each terminal (e.g., 210 in FIG. 2A) is only coupled to one electrode 134.

The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. The one or more lumens may, optionally, be flushed continually, or on a regular basis, with saline, epidural fluid, or the like. The one or more lumens can be permanently or removably sealable at the distal end.

As discussed above, the one or more lead bodies 106 may be coupled to the one or more connector assemblies 144 disposed on the control module 102. The control module 102 can include any suitable number of connector assemblies 144 including, for example, two three, four, five, six, seven, eight, or more connector assemblies 144. It will be understood that other numbers of connector assemblies 144 may be used instead. In FIG. 1, each of the two lead bodies 106 includes eight terminals that are shown coupled with eight conductive contacts disposed in a different one of two different connector assemblies 144.

FIG. 2A is a schematic side view of one embodiment of the two lead bodies 106 shown in FIG. 1 configured and arranged for coupling with the control module 102. A plurality of connector assemblies 144 are disposed in the control module 102. In at least some embodiments, the control module 102 includes two, three, four, or more connector assemblies 144. Typically, the number of connector assemblies 144 disposed in the control module 102 is equal to the number of lead bodies 106 of the lead. For example, in FIG. 2A, the two lead bodies 106 shown in FIG. 1 are shown configured and arranged for insertion into two connector assemblies 144 disposed on the control module 102.

The connector assemblies 144 each include a connector housing 214 and a plurality of connector contacts 316 disposed therein. Typically, the connector housing 214 defines a port (not shown) that provides access to the plurality of connector contacts 216. In at least some embodiments, the connector assemblies 144 further include retaining elements 218 configured and arranged to fasten the corresponding lead bodies 206 to the connector assemblies 144 when the lead bodies 106 are inserted into the connector assemblies 144 to prevent undesired detachment of the lead bodies 106 from the connector assemblies 144. For example, the retaining elements 218 may include apertures through which fasteners (e.g., set screws, pins, or the like) may be inserted and secured against an inserted lead body (or lead extension).

In FIG. 2A, the plurality of connector assemblies 144 are disposed in the header 150. In at least some embodiments, the header 150 defines one or more ports 204 into which a proximal end 206 of the one or more lead bodies 106 with terminals 210 can be inserted, as shown by directional arrows 212, in order to gain access to the connector contacts 216 disposed in the connector assemblies 144.

When the lead bodies 106 are inserted into the ports 204, the connector contacts 216 can be aligned with the terminals 210 disposed on the lead bodies 106 to electrically couple the control module 102 to the electrodes (134 of FIG. 1) disposed at a distal end of the lead bodies 106. Examples of connector assemblies in control modules are found in, for example, U.S. Pat. Nos. 7,244,150 and 8,224,450, which are incorporated by reference.

In some instances, the electrical stimulation system may include one or more lead extensions. FIG. 2B is a schematic side view of one embodiment of a proximal end of a single lead body 106′ configured and arranged to couple with a lead extension 224 that is coupled with the control module 102′. In FIG. 2B, a lead extension connector assembly 222 is disposed at a distal end 226 of the lead extension 224. The lead extension connector assembly 222 includes a contact housing 228. The contact housing 228 defines at least one port 230 into which a proximal end 206 of the lead body 106′ with terminals 210 can be inserted, as shown by directional arrow 238. The lead extension connector assembly 222 also includes a plurality of connector contacts 240. When the lead body 106′ is inserted into the port 230, the connector contacts 240 disposed in the contact housing 228 can be aligned with the terminals 210 on the lead body 106 to electrically couple the lead extension 224 to electrodes (not shown) disposed on the lead body 106′.

The proximal end of a lead extension can be similarly configured and arranged as a proximal end of a lead body, such as one of the lead bodies 106, or the lead body 106′. The lead extension 224 may include a plurality of conductive wires (not shown) that electrically couple the connector contacts 240 to terminals at the proximal end 248 of the lead extension 224. The conductive wires disposed in the lead extension 224 can be electrically coupled to a plurality of terminals (not shown) disposed on the proximal end 248 of the lead extension 224.

FIG. 2C is a schematic side view of one embodiment of the lead extension 224 configured and arranged for coupling with the control module 102′. The control module 102′ includes a single connector assembly 144. Alternately, the control module 102′ may receive the lead body 106′ directly. It will be understood that the control modules 102 and 102′ can both receive either lead bodies or lead extensions. It will also be understood that the electrical stimulation system 100 can include a plurality of lead extensions 224. For example, each of the lead bodies 106 shown in FIGS. 1 and 2A can, alternatively, be coupled to a different lead extension 224 which, in turn, are each coupled to different ports of a two-port control module, such as the control module 102 of FIGS. 1 and 2A.

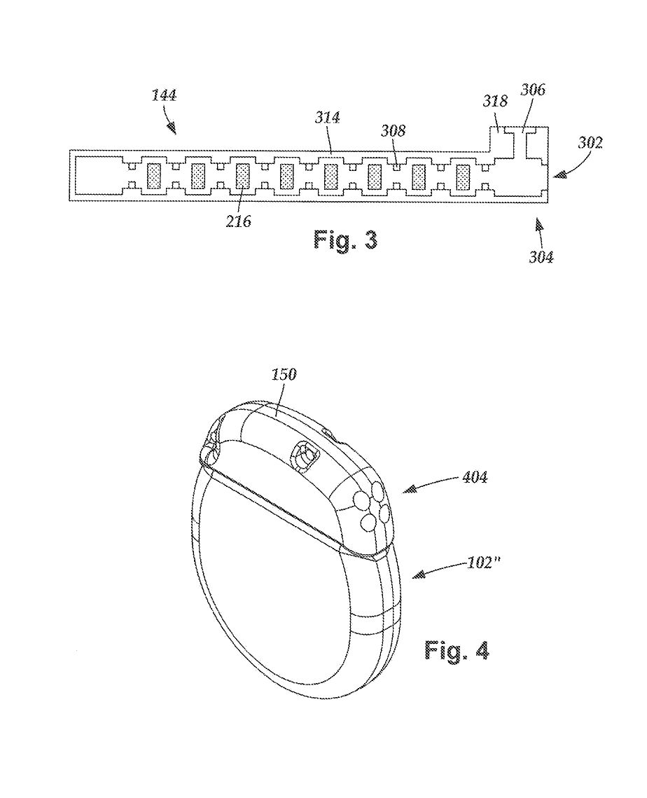

FIG. 3 is a schematic longitudinal cross-sectional view of one embodiment of one of the connector assemblies 144. The connector assembly 144 includes the connector housing 314 into which a lead or lead extension can be inserted via a port 302 at a distal end 304 of the connector housing 314. In at least some embodiments, a retaining element 318 is coupled to the connector housing 314. The retaining element 318 defines an aperture 306 through which a fastener (e.g., a set screw, pin, or the like) may be inserted and secured against a lead body or lead extension when the lead or lead extension is inserted into the port 302. Connector contacts, such as the connector contact 216, are disposed in the connector housing 314. In at least some embodiments, each of the connector assemblies 144 includes eight connector contacts.

The connector contacts 216 may be separated from one another by one or more non-conductive spacers (or seals), such as spacer 308, to prevent electrical contact between adjacent connector contacts 216. As discussed above, when a proximal end of a lead or lead extension is inserted into the port 302, terminals disposed on the inserted lead or lead extension align with the connector contacts 216, thereby establishing an electrical connection between the electronic subassembly 110 of the control module 102 and the electrodes 134 of the lead.

FIG. 4 is a schematic perspective view of a control module 102″. The header 150 of the control module 102″ defines four header ports 404. Collectively, the header ports 404 are configured and arranged to each receive one or more lead bodies 106 or one or more lead extensions (e.g., lead extension 324 of FIG. 3B), or both. The header 150 can define any suitable number of header ports 404 including, for example, one, two, three, four, five, six, seven, eight, or more header ports 404. In FIG. 4, the header 150 is shown defining four header ports 404. Thus, in at least some embodiments, the control module 102 of FIG. 4 is configured and arranged to receive up to four lead bodies 106 or lead extensions 224, or a combination of both.

The header ports 404 can be defined in the header 150 in any suitable arrangement. In preferred embodiments, each of the header ports 404 are configured and arranged to align with one of the ports 302 of the one or more connector assemblies 144 disposed in the header 150. For example, in at least some embodiments, four connector assemblies 144 are disposed in the header 150 such that four header ports 404 defined in the header 150 align with the four ports 302 of the four connector assemblies 144. In at least some embodiments, the number of header ports 404 is no greater than the number of connector assemblies 144. In at least some embodiments, the number of header ports 404 is no less than the number of connector assemblies 144. In at least some embodiments, the number of header ports 404 is equal to the number of connector assemblies 144.

Paddle bodies are typically implanted into patients with the electrodes of the paddle bodies in close proximity to the patient tissue to be stimulated. During operation, it may be desirable for the implanted paddle bodies to maintain a constant positioning relative to the tissue being stimulated to maintain a therapeutic effect. Paddle bodies, however, may be vulnerable to being jostled during operation due to, for example, ordinary patient movement. Jostling of the paddle body may lead to dislodgement of the paddle body from an implantation location, and, potentially, a loss of therapeutic effect.

Conventional paddle bodies may be particularly vulnerable to dislodgement due to the limited ability of the paddle bodies to flex when a force is applied to the paddle bodies during, for example, patient movement. The limited flexibility of the paddle bodies may be caused, at least in part, by the electrodes disposed along a major surface of the paddle body. Electrodes are typically formed from materials that are significantly less flexible than materials forming the paddle body itself. In which case, the flexibility of the paddle body may be limited, at least in part, by the sizes, shapes, and arrangements of the electrodes along the paddle body. Additionally, since increasing the surface areas of electrodes may reduce the current density of the electrodes, there is at least some incentive to form the electrodes with larger sizes, thereby potentially reducing the flexibility of the electrodes.

At least some conventional paddle bodies include an array of generally rectangular electrodes arranged into columns that extend parallel to a longitudinal axis of the paddle body. In which case, flexibility of the paddle body may be limited to one or more lines of flexure extending along the longitudinal axis of the paddle body between adjacent columns of electrodes.

During implantation, paddle leads may pass through tortuous anatomy during advancement to a target implantation location. When, as with conventional paddle leads, flexibility of the paddle bodies are limited to one or more lines of flexure extending along the longitudinal axes of paddle bodies between adjacent columns of electrodes, advancement of the paddle leads to the target implantation location may cause pain or discomfort to the patient. In some cases, patient tissue may need to be cut in order to enable passage of the paddle lead.

As herein described, a paddle lead assembly includes five-sided electrodes arranged along a paddle body such that each of the electrodes abuts at least one other electrode along one or more interfaces that extend in directions that are parallel to neither a longitudinal axis nor a transverse axis of the paddle body. In at least some embodiments, each of the electrodes also abuts at least one other electrode along one or more interfaces that extend in directions that are parallel to one or more of the longitudinal axis or the transverse axis of the paddle body.

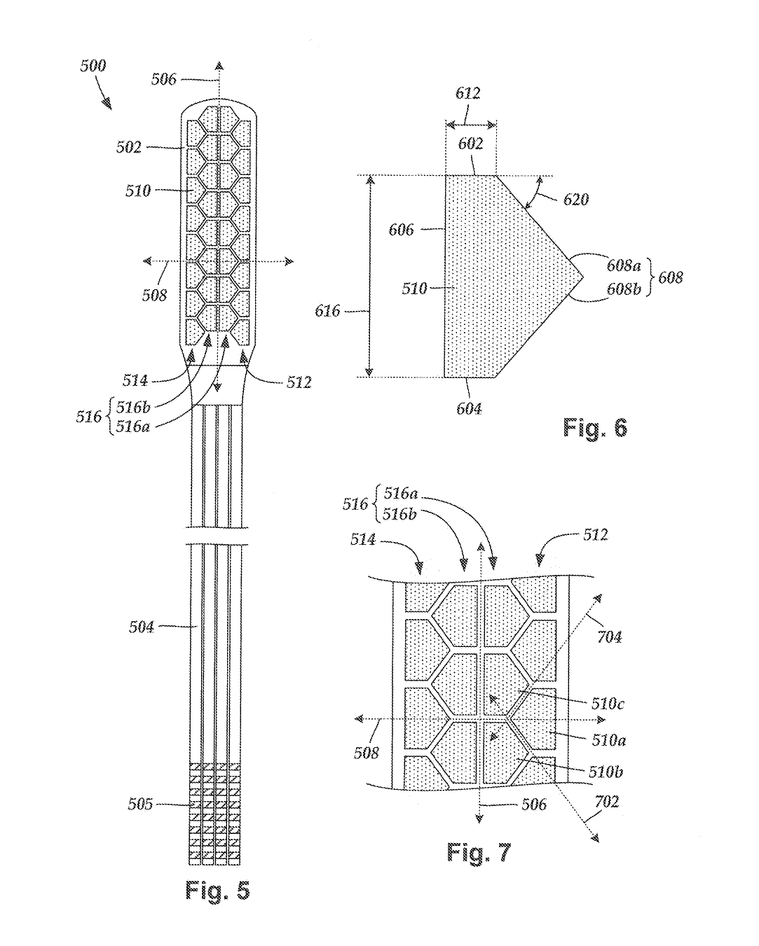

FIG. 5 is a top schematic view of one embodiment of a paddle lead assembly 500. The paddle lead assembly 500 includes a paddle body 502 and a plurality of lead bodies, such as lead body 504. At least one of the plurality of lead bodies 504 includes a plurality of terminals, such as terminal 505. In at least some embodiments, at least one of the plurality of terminals 505 is disposed on each of the plurality of lead bodies 504. For example, in FIG. 5 eight terminals 505 are shown disposed along each of four lead bodies 504. It will be understood that the paddle lead assembly 500 can include any suitable number of lead bodies 504 including, for example, one, two, three, four, five, six, seven, eight, or more lead bodies 504.

The paddle body 502 includes a longitudinal axis 506 and a transverse axis 508 that is transverse to the longitudinal axis 506. The paddle body 502 can have any suitable length along the transverse axis 508 (i.e., width). In at least some embodiments, the paddle body 502 has a width (i.e., along the transverse axis 508) of no more than 12 mm, 11 mm, 10 mm, 9 mm, 8 mm, or less. The paddle body 502 includes an array of electrodes, such as electrode 510. The array can include any number of electrodes 510 including, for example, sixteen, eighteen, twenty, twenty-two, twenty-four, twenty-six, twenty-eight, thirty, thirty-two, thirty-four, forty, forty-eight, or more electrodes. It will be understood that other numbers of electrodes 510 may be used instead.

The electrodes 510 may be arranged into columns extending parallel with the longitudinal axis 506 of the paddle body 502. In at least some embodiments, the columns of electrodes include lateral columns 512 and 514 and one or more medial columns 516. In FIG. 5, the paddle body 502 is shown with two medial columns 516a and 516b. In at least some embodiments, the medial columns 516a and 516b of electrodes 510 are staggered longitudinally relative to the lateral columns 512 and 514 of electrodes 510 such that there is no single transverse axis extending across the paddle body 502 that passes through a center of an electrode 510 of each of the columns 512, 514, 516 of the electrodes 510.

Each of the columns 512, 514, 516 of the electrode array 510 may include the same number of electrodes 510. In at least some embodiments, at least one of the columns 512, 514, 516 of electrodes 510 includes a different number of electrodes 510 from one or more of the other columns 512, 514, 516 of electrodes 510. In at least some embodiments, each of the lateral columns, 512 and 514 includes the same number of electrodes. In at least some embodiments with two or more medial columns of electrodes, each of the two or more medial columns 516 includes the same number of electrodes 510. In at least some embodiments, the total number of electrodes 510 disposed in the lateral columns 512, 514 is equal to the total number of electrodes 510 disposed in the one or more medial columns 516. In at least some embodiments with two or more medial columns of electrodes, each of the lateral columns 512 and 514 includes the same number of electrodes 510, and each of the two or more medial columns 516 also includes the same number of electrodes 510, where the number of electrodes 510 disposed in the lateral columns 512 and 514 is different from the number of electrodes 510 disposed in the two or more medial columns 516.

In FIG. 5, each of the columns 512, 514, 516 of electrodes 510 is shown having eight electrodes. It will be understood that other numbers of electrodes, either fewer or greater, may be disposed in each column. For example, each of the columns 512, 514, 516 can include two, three, four, five, six, seven, eight, nine, ten, eleven, twelve, thirteen, fourteen, fifteen, sixteen, seventeen, eighteen, nineteen, twenty, twenty-four, thirty-two or more electrodes 510.

Each of the electrodes 510 may be independently operated, via a pulse generator disposed in the control module (102 in FIG. 1). In at least some embodiments, the control module 102 has at least as many independently programmable stimulation channels as electrodes 510. In at least some embodiments, the control module 102 stimulation channels are independently programmable, preferably to independently deliver constant current stimulus pulses to any one or more of the electrodes 510.

Turning back to FIGS. 3 and 4 (in combination with FIG. 5), in at least some embodiments four connector assemblies 144 are disposed in the header 150 and accessible to up to four lead bodies 505 via four header ports 404. Each port 302 defined in the connector assemblies 144 can be configured and arranged to enable an electrical connection between the terminals 505 of the lead bodies 504. In at least some embodiments, each port (302 of FIG. 3) of the connector assemblies (144 of FIG. 3) has eight connector contacts. In at least some embodiments, the control module (102″ in FIG. 4) has a total of 32 independently programmable stimulation channels.

In at least some embodiments with two or more medial columns 516, at least some of the electrodes 516 of the two or more medial columns 516 may be longitudinally-aligned with one another such that the electrodes of those columns form rows along the transverse axis 508 such that a single transverse axis extending across the paddle body 502 passes through a center of an electrode 510 of each of the two or more medial columns 516.

In at least some embodiments, at least some of the longitudinally-aligned electrodes 510 of the two or more medial columns 516 are configured and arranged to operate as anodes. In at least some embodiments, electrodes of the lateral columns 512, 514 are also longitudinally-aligned with one another such that a single transverse axis extending across the paddle body 502 passes through a center of an electrode 510 of each of the lateral columns 512, 514. In at least some embodiments, at least some of the longitudinally-aligned electrodes of the lateral columns 512, 514 are configured and arranged to operate as cathodes.

The electrodes of the electrode array 510 can have any suitable center-to-center spacing between adjacent electrodes in a given column, or longitudinal spacing. In at least some embodiments, the longitudinal spacing between adjacent electrodes 510 is no greater than 7 mm, 6.5 mm, 6 mm, 5.5 mm, 5 mm, 4.5 mm, or less. It will be understood that all longitudinal spacings between adjacent electrodes are measured as center-to-center distances.

In at least some embodiments, each of the electrodes of the lateral column 512 are equally spaced apart longitudinally from one another (i.e., the adjacent electrodes have equal longitudinal spacings). In at least some embodiments, each of the electrodes of the lateral column 514 are equally spaced apart longitudinally from one another (i.e., the adjacent electrodes have equal longitudinal spacings). In at least some embodiments, the longitudinal spacing of adjacent electrodes of the lateral column 512 is equal to the longitudinal spacing of adjacent electrodes of the lateral column 514. In at least some embodiments, each of the electrodes of the medial column 516a are equally spaced apart longitudinally from one another (i.e., the adjacent electrodes have equal longitudinal spacings). In at least some embodiments, each of the electrodes of the medial column 516b are equally spaced apart longitudinally from one another (i.e., the adjacent electrodes have equal longitudinal spacings). In at least some embodiments, the longitudinal spacing of adjacent electrodes of the medial column 516a is equal to the longitudinal spacing of adjacent electrodes of the medial column 516b. In at least some embodiments, the longitudinal spacing of adjacent electrodes of the lateral column 512 is equal to the longitudinal spacing of adjacent electrodes of each of the lateral column 514, the medial column 516a, and the medial column 516b. In at least some embodiments, the transverse spacing between electrodes of the lateral column 512 and electrodes of the medial column 516a is equal to the transverse spacing between electrodes of the lateral column 514 and the medial column 516b.

The electrodes can be configured into any number of columns greater than two, and any number of electrodes can be disposed in any of the columns. When the paddle lead assembly 500 includes thirty-two electrodes, many different electrode configurations are possible including, for example, two lateral columns with six electrodes each, two medial columns with six electrodes, and one medial column with eight electrodes. This configuration may simply be referred to as 6-6-8-6-6, where the number of electrodes in a column are counted from left to right. It may be helpful to use this shorthand notation. Alternately, the above configuration may be rearranged such that the eight-electrode column may be disposed in the second position (6-8-6-6-6), or the fourth position (6-6-6-8-6). Other 32-electrode configurations may include, for example 6-7-6-7-6; 5-5-6-6-5-5; 4-4-5-6-5-4-4; and 4-4-4-4-4-4-4-4. It will be understood that other rearrangements and configurations are possible, as well.

Turning now to FIG. 6, in at least some embodiments the electrodes are each five-sided. In at least some embodiments, the five-sided electrodes may have a larger surface area than electrodes of conventional paddle lead assemblies. Increasing surface area of the electrodes may provide the benefit of reducing the current density. Additionally, in at least some embodiments the five-sided electrodes can be arranged such that the current density is reduced without the potentially deleterious effects of decreasing flexibility of the paddle lead assembly. The five-sided electrode configuration enables the electrodes to abut one another along a plurality of different interfaces that may improve the ability of the paddle lead to flex along a variety of different angles. In which case, the five-sided electrodes may enable the paddle body to twist with patient movement, thereby potentially reducing the potential for dislodgement of the paddle body due to patient movement. Additionally, the twistability of the five-sided electrodes may improve implantation of the paddle lead by facilitating advancement of the paddle body through tortuous anatomy, thereby potentially reducing patient pain and discomfort associated with advancement of the paddle body to the target implantation location.

FIG. 6 is a schematic top view of one embodiment of the five-sided electrode 510 suitable for use with the paddle lead assembly (500 in FIG. 5). The electrode 510 includes a first end 602; a second end 604 opposite to the first end 602; a single-sided face 606 extending between the first end 602 and the second end 604; and a double-sided face 608 opposite to the single-sided face 606, the double-sided face 608 including a first side 608a and a second side 608b, where the first side 608a extends between the first end 602 and the second side 608b, and the second side 608b extends between the second end 604 and the first side 608a.

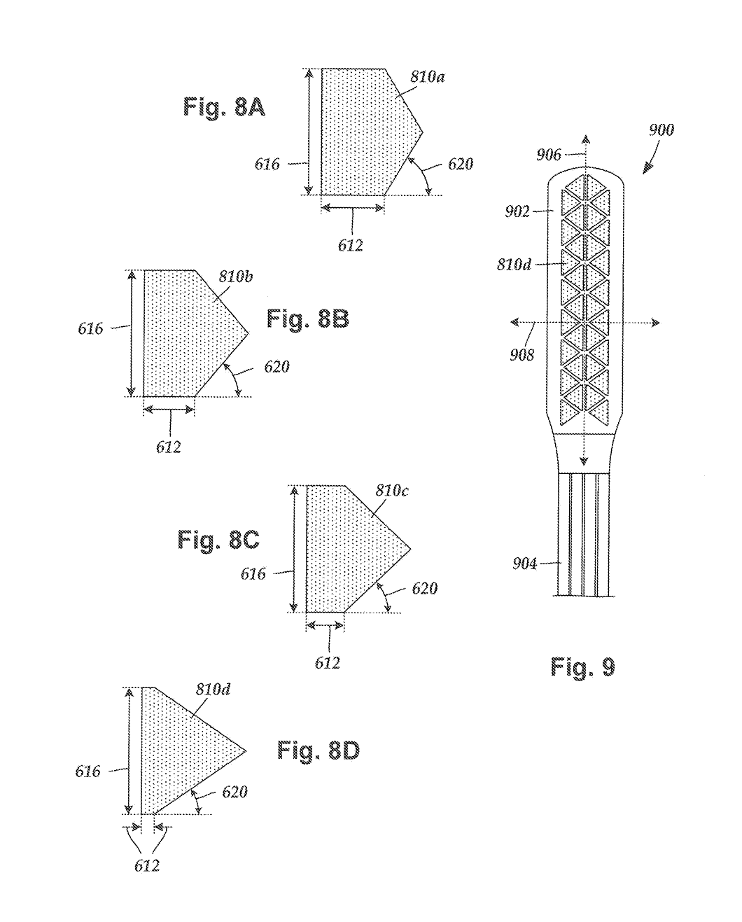

In at least some embodiments, the first end 602 and the second end 604 are parallel with one another. The first end 602 has a length 612. In at least some embodiments, the second end 604 has a length that is equal to the length 612 of the first end 602. The single-sided face 606 has a length 616. As discussed in more detail below with respect to FIGS. 8A-8D, the lengths 612 and 616 can vary relative to one another.

In at least some embodiments, the single-sided face 606 extends perpendicularly to at least one of the first end 602 and the second end 604. An angle 620 is formed between an axis extending along the first end 602 and the first side 608a. As discussed below with reference to FIG. 7, when a plurality of the electrodes 510 are disposed on the paddle body 502 (see e.g., FIG. 5) the angle 620 determines an angle of an interface between two adjacent electrodes. The first side 608a and the second side 608b can have any suitable lengths. In some embodiments, the first side 608a and the second side 608b have equal lengths. In other embodiments, the first side 608a and the second side 608b have different lengths.

When the lead body is more flexible than the electrodes disposed on the lead body, the interface forms line of flexure along the paddle body 502 between the two adjacent electrodes 510. The angle 620 is greater than zero and less than 90°. In at least some embodiments, the angle 620 is no less than 20° and no greater than 70°. In at least some embodiments, the angle 620 is no less than 30° and no greater than 60°. In at least some embodiments, the angle 620 is no less than 40° and no greater than 50°. In at least some embodiments, the angle 620 is 45°.

In at least some embodiments the electrodes 510 are arranged along the paddle body 502 such that at least some interfaces between adjacent electrodes (and the resulting lines of flexure formed between those electrodes) is neither parallel to the longitudinal axis 506 nor the transverse axis 508 of the paddle body 502. The angle 620 can be varied, as desired, depending on, for example, the size and shape of the paddle body 502, the number of electrodes 510 disposed on the paddle body 502, the anticipated axes of flexing of the paddle body 502 during operation, or the like.

FIG. 7 is a schematic top view of a plurality of the five-sided electrodes 510 disposed along a portion of the paddle body 502. As shown in FIG. 7, the electrodes 510 are disposed into lateral columns 512 and 514 and two medial columns 516a and 516b, where each electrode 510 within a given column is oriented in the same direction. In at least some embodiments, each electrode 510 within a given column is oriented in the same direction, and also oriented opposite to each of the electrodes in each adjacent column. As shown in FIGS. 5 and 7, in at least some embodiments the electrodes 510 of the lateral columns 512 and 514 are each oriented such that the single-sided faces 606 extend away from the medial columns 516a and 51b, while the electrodes of the medial columns 516a and 516b are oriented such that the single-sided faces 606 of the medial column 516a abut the single-sided faces 606 of the medial column 516b.

In at least some embodiments, the electrodes 510 of the medial columns 516a and 516b are aligned with one another along the longitudinal axis 506 of the paddle body 502 and the lateral columns 512 and 514 are aligned with one another along the longitudinal axis 506 of the paddle body 502 such that the lateral columns 512 and 514 are longitudinally offset from the medial columns 516a and 516b along the longitudinal axis 506 of the paddle body 502.

Thus, in at least some embodiments the electrodes 510 are interleaved along the paddle body 502 such that the double-sided faces 608 of the electrodes 510 of the lateral columns 512 and 514 abut the double-sided faces 608 of the electrodes 510 of the medial columns 516a and 516b, respectively. In FIG. 7, one side of the double-sided face of the electrode 510a of the lateral column 512 is shown abutting one side of the double-sided face of the electrode 510b of the medial column 516a. The interface between abutting sides of the electrode 510a and 510b forms a first line of flexure 702 along the paddle body 502 between the electrodes 510a and 510b. As mentioned above, the interface between the abutting sides of the electrodes 510a and 510b form a first line of flexure 702 of the paddle body 502 because the paddle body 502 is typically formed from one or more materials that are more flexible than the one or more materials used to form the electrodes 510. Thus, the paddle body 502 is more likely to flex along regions of the paddle body 502 that are not covered by one of the electrodes 502, such as the first line of flexure 702 extending between the electrode 510a and the electrode 510b.

In at least some embodiments, the double-sided regions of at least some of the electrodes abut the double-sided region of two other electrodes. In FIG. 7, in addition to abutting the double-sided region of the electrode 510b, the double-sided region of the electrode 510a also abuts one side of the double-sided region of electrode 510c to form a second line of flexure 704 along the paddle body 502. As also shown in FIG. 7, neither of the lines of flexure 702, 704 formed by the interfaces between double-sided regions of abutting electrodes is parallel with either the longitudinal axis 506 or the lateral axis 508 of the paddle body 502.

In at least some embodiments, the first line of flexure 702 and the second line of flexure 704 are equal to one another with respect to the transverse axis 508 of the paddle body 502. Each of the first line of flexure 702 and the second line of flexure 704 can be any suitable angle with respect to the transverse axis 508 including, for example, 5°, 10°, 15°, 20°, 25°, 30°, 35°, 40°, 45°, 50°, 55°, 60°, 65°, 70°, 75°, 80°, or 85°. In at least some embodiments, the first line of flexure 702 and the second line of flexure 704 each form an angle with respect to the transverse axis 508 that is no less than 45°. In at least some embodiments, the first line of flexure 702 and the second line of flexure 704 each form an angle with respect to the transverse axis 508 that is no greater than 45°. In at least some embodiments, the first line of flexure 702 and the second line of flexure 704 each form an angle with respect to the transverse axis 508 that is no less than 20° and no greater than 70°. In at least some embodiments, the first line of flexure 702 and the second line of flexure 704 each form an angle with respect to the transverse axis 508 that is no less than 30° and no greater than 60°. In at least some embodiments, the first line of flexure 702 and the second line of flexure 704 each form an angle with respect to the transverse axis 508 that is no less than 40° and no greater than 50°. In at least some embodiments, the first line of flexure 702 and the second line of flexure 704 each form an angle with respect to the transverse axis 508 that is equal to 45°.

In at least some embodiments, the electrode may be disposed on the paddle body such that one or more lines of flexure are formed that are parallel with the longitudinal axis 506. For example, in FIG. 7 the electrodes 510 of the medial columns 516a and 516b are oriented such that the single-sided faces (606 in FIG. 6) of the electrodes 510 of the medial columns 516a and 516b abut one another along the longitudinal axis 506 of the paddle body 502, thereby forming a line of flexure parallel with the longitudinal axis 506.

In at least some embodiments, the electrode may be disposed on the paddle body such that one or more lines of flexure are formed that are parallel with the transverse axis 508. For example, in FIG. 7 the electrodes of the lateral columns 512, 514 and medial columns 516a, 516b are oriented such that the first ends (602 in FIG. 6) and the opposing second ends (604 in FIG. 6) of the electrodes of the lateral columns 512, 514 and medial columns 516a, 516b are longitudinally-aligned with one another along the longitudinal axis 506 of the paddle body 502, thereby forming lines of flexure along the paddle body 502 that are parallel with the lateral axis 508.

Turning to FIGS. 8A-8D, the first and second ends of the electrodes can have any suitable lengths 612. The single-sided faces of the electrodes can also have any suitable lengths 616. The angle 620 formed between the axis extending along the first end 602 and the first side 608a can be any suitable angle between 0° and 90°.

In each of FIGS. 8A-8D, the first end and the second end of the electrodes are shown having the same length. It will be understood that, in alternate embodiments, the first end and the second end have different lengths from one another. In FIGS. 8A-8D, the lengths 612 of each of the first and second ends are less than the lengths 616 of the single-sided faces. It will be understood that, in alternate embodiments, for at least one of the electrodes 510 the length 612 of at least one of the first and second ends is greater than the length 616 of the single-sided face.

FIG. 8A is a schematic view of a first exemplary embodiment of an electrode 810a suitable for use with the paddle body assembly (500 in FIG. 5). In FIG. 8A, the length 612 of each of the first and second ends is no greater than 50% of the length 616 of the single-sided face. FIG. 8B is a schematic view of a second exemplary embodiment of the electrode 810b suitable for use with the paddle body assembly (500 in FIG. 5). In FIG. 8B, the length 612 of each of the first and second ends is no greater than 40% of the length 616 of the single-sided face. In FIGS. 8A and 8B, the angle 620 is shown being less than 90° and no less than 45°.

FIG. 8C is a schematic view of a third exemplary embodiment of the electrode 810c suitable for use with the paddle body assembly (500 in FIG. 5). In FIG. 8C, the length 612 of each of the first and second ends is no greater than 30% of the length 616 of the single-sided face. FIG. 8D is a schematic view of a fourth exemplary embodiment of the electrode 810d suitable for use with the paddle body assembly (500 in FIG. 5). In FIG. 8D, the length 612 of each of the first and second ends is no greater than 10% of the length 616 of the single-sided face. In at least some embodiments where the length 612 of each of the first and second ends is each no greater than 10% of the length 616 of the single-sided face, the shape of the electrode is substantially triangular. In FIGS. 8C and 8D, the angle 620 is shown being greater than 0° and no more than 45°.

Turning to FIG. 9, any of the embodiments of the electrodes shown in FIGS. 8A-8D can be disposed on the paddle lead assembly. FIG. 9 is a schematic top view of another embodiment of a paddle lead assembly 900. The paddle lead assembly 900 includes a paddle body 902 and a plurality of lead bodies, such as lead body 904 coupled to the paddle body 902. The paddle body 902 includes a longitudinal axis 906 and a transverse axis 908 that is transverse to the longitudinal axis 906. A plurality of the electrodes 810d are disposed on the paddle body 902 in a configuration that is similar to the configuration shown in FIG. 5.

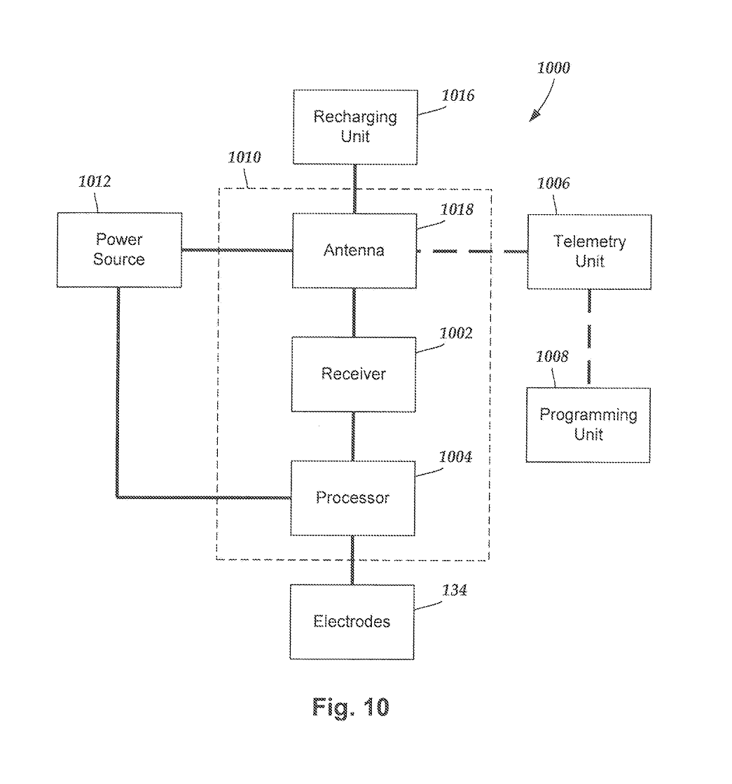

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A paddle lead assembly for providing electrical stimulation of patient tissue, the paddle lead comprising:
a paddle body having a longitudinal axis and a transverse axis transverse to the longitudinal axis;
a plurality of electrodes disposed along the paddle body, each of the plurality of electrodes having a five-sided shape;
at least one lead body coupled to the paddle body;
a plurality of terminals disposed on the at least one lead body; and
a plurality of conductive wires, each conductive wire coupling one of the plurality of terminals to at least one of the plurality of electrodes.

2. The paddle lead assembly of claim 1, wherein for each of the plurality of electrodes the five-sided shape comprises a first end, a second end opposite and parallel to the first end, a single-sided face extending between the first end and the second end, and a double-sided face opposite to the single-sided face and also extending between the first end and the second end.

3. The paddle lead assembly of claim 2, wherein for each of the plurality of electrodes the double-sided face comprises a first side and a second side, the first side extending between the first end and the second side, the second side extending between the second end and the first side.

4. The paddle lead assembly of claim 3, wherein for each of the plurality of electrodes the first side and the second side of the double-sided face have equal lengths.

5. The paddle lead assembly of claim 3, wherein for each of the plurality of electrodes, at least one of the first side or the second side of the double-sided face of the electrode abuts at least one of the first side or the second side of the double-sided face of another electrode of the plurality of electrodes to form an interface that extends along an axis that is parallel with neither the longitudinal axis nor the lateral axis of the paddle body.

6. The paddle lead assembly of claim 2, wherein for each of the plurality of electrodes the single-sided face is perpendicular to both the first end and the second end.

7. The paddle lead assembly of claim 2, wherein for each of the plurality of electrodes the first end and the second end have equal lengths.

8. The paddle lead assembly of claim 2, wherein for each of the plurality of electrodes the single-sided face has a length that is at least two times the length of either the first end or the second end.

9. The paddle lead assembly of claim 2, wherein the plurality of electrodes are arranged into a plurality of columns, the plurality of columns comprising a first column and second column adjacent to the first column along the transverse axis of the paddle body, each of the plurality of columns comprising at least two different electrodes of the plurality of electrodes.

10. The paddle lead assembly of claim 9, wherein each of the at least two electrodes of the first column are arranged in a first orientation and each of the at least two electrodes of the second column are arranged in a second orientation opposite to the first orientation, wherein for each of the electrodes of the first column at least a portion of the double-sided face of the electrode abuts at least a portion of the double-sided face of at least one of the at least two electrodes of the second column.

11. The paddle lead assembly of claim 9, wherein a center of each of the at least two electrodes of the first column is offset along the longitudinal axis of the paddle body from a center of each of the at least two electrodes of the second column.

12. The paddle lead assembly of claim 9, wherein the at least two electrodes of the first column and the at least two electrodes of the second column have the same longitudinal center-to-center spacing between adjacent electrodes.

13. The paddle lead assembly of claim 12, wherein a center of each of the at least two electrodes of the first column is offset along the longitudinal axis of the paddle body from a center of each of the at least two electrodes of the second column by half of the longitudinal center-to-center spacing between adjacent electrodes.

14. The paddle lead assembly of claim 9, wherein the first column and the second column each comprise eight electrodes.

15. The paddle lead assembly of claim 9, wherein the plurality of electrodes are arranged into four columns of electrodes with each of the four columns comprising at least two electrodes.

16. An electrical stimulating system comprising:
the paddle lead assembly of claim 1;
at least one control module configured and arranged to electrically couple to each of the electrodes, each of the at least one control module comprising
a housing, and
an electronic subassembly disposed in the housing, the electronic subassembly comprising a pulse generator; and
a connector assembly for receiving the at least one lead body, the connector assembly comprising
a connector housing defining at least one port at a distal end of the connector housing, the at least one port configured and arranged for receiving the at least one lead body of the paddle lead assembly, and
at least one connector contact disposed in the connector housing, the at least one connector contact configured and arranged to couple to at least one of the plurality of terminals disposed on the at least one lead body of the paddle lead assembly.

17. A method for stimulating patient tissue, the method comprising:
inserting the paddle lead assembly of claim 1 into a patient;
inserting a portion of at least one lead body of the paddle lead assembly into a connector assembly coupled to a control module, the connector assembly comprising a connector housing defining at least one port at a distal end of the connector housing and at least one connector contact disposed in the connector housing, the connector contact configured and arranged to couple to at least one of the plurality of terminals disposed on the at least one lead body of the paddle lead assembly, the control module comprising a housing and an electronic subassembly disposed in the housing, the electronic subassembly comprising a pulse generator;
generating electrical signals using the pulse generator; and
providing the generated electrical signals to the plurality of electrodes of the paddle lead assembly.

18. A paddle lead assembly for providing electrical stimulation of patient tissue, the paddle lead comprising:
a paddle body having a longitudinal axis and a transverse axis transverse to the longitudinal axis;
a plurality of five-sided electrodes disposed along the paddle body with each electrode of the plurality of electrodes comprising a first edge portion and a second edge portion, wherein for each electrode of the plurality of electrodes the first edge portion abuts the first edge portion of another electrode of the plurality of electrodes along a first interface, and wherein the first interface extends in a direction that is parallel to neither the longitudinal axis nor the transverse axis of the paddle body;
at least one lead body coupled to the paddle body;
a plurality of terminals disposed on the at least one lead body; and
a plurality of conductive wires, each conductive wire coupling one of the plurality of terminals to at least one of the plurality of electrodes.

19. The paddle lead assembly of claim 18, wherein each electrode of the plurality of electrode abuts at least two other electrodes of the plurality of electrodes.

20. The paddle lead assembly of claim 18, wherein for each electrode of the plurality of electrodes the second edge portion abuts the second edge portion of another electrode of the plurality of electrodes along a second interface, and wherein the second interface extends in a direction that is parallel to one of the longitudinal axis or the transverse axis of the paddle body.
